(19) United States
(12) Patent Application Publication
(10) Pub. No.: US 2025/0298014 A1
(43) Pub. Date: Sep. 25, 2025

(54) A MOLECULAR SENSOR AND METHODS FOR USE

(71) Applicant: Roswell ME, Inc., San Diego, CA (US)

(72) Inventors: Carl Fuller, Berkeley Heights, NJ (US); Barry Merriman, San Diego, CA (US); Paul W. Mola, San Diego, CA (US)

(73) Assignee: Roswell ME, Inc., San Diego, CA (US)

(21) Appl. No.: 18/709,785

(22) PCT Filed: Nov. 14, 2022
(86) PCT No.: PCT/US2022/049869

§ 371 Date: May 13, 2024

Related U.S. Application Data
(60) Provisional application No. 63/279,055, filed on Nov. 12, 2021.

(51) Int. Cl. G01N 33/543; G01N 33/53; G01N 33/569

(52) U.S. Cl. CPC G01N 33/5438; G01N 33/5308; G01N 33/56983; G01N 2333/165; G01N 2469/10

(57) ABSTRACT

In various embodiments a molecular circuit is disclosed. The circuit comprises a negative electrode, a positive electrode spaced apart from the negative electrode, and an enzyme molecule conductively attached to both the positive and negative electrodes to form a circuit having a conduction pathway through the enzyme. In various examples, the enzyme is a polymerase. The circuit may further comprise molecular arms used to wire the enzyme to the electrodes. In various embodiments, the circuit functions as a sensor, wherein electrical signals, such as changes to voltage, current, impedance, conductance, or resistance in the circuit, are measured as substrates interact with the enzyme.

### A MOLECULAR SENSOR AND METHODS FOR USE

The disclosure claims priority to PCT Application Serial No. PCT/US2022/049869, filed Nov. 14, 2022, which itself claims priority to the U.S. Provisional Application Ser. No. 63/279,055, filed Nov. 12, 2021; the specification of the foregoing applications are incorporated herein in their entirety.

### FIELD

The present disclosure is generally directed to molecular sensors and more particularly to molecular sensors in which an enzyme closes the circuit between two electrodes.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

The broad field of molecular electronics was introduced in the 1970's by Aviram and Ratner. Molecular electronics achieves the ultimate scaling down of electrical circuits by using single molecules as circuit components. Molecular circuits comprising single molecule components can function diversely as switches, rectifiers, actuators and sensors, depending on the nature of the molecule. Of particular interest is the application of such circuits as sensors, where molecular interactions provide a basis for single molecule sensing. In particular, informative current changes could include an increase, or decrease, a pulse, or other time variation in the current.

Notwithstanding the achievements in the field of molecular electronics, new molecular circuits that can function as molecular sensors are still needed. In particular, for new molecular circuits that can detect a wide range of ligands. The need still also exists for improved single molecule systems that can yield molecular information with greater signal-to-noise ratios such that signals truly indicative of molecular interactions are distinguishable from non-informative noise. The inventions described herein meet these unsolved challenges and needs. As described in detail below, novel embodiments of the invention described herein.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

In various embodiments, single molecule enzyme-based circuits are disclosed wherein a single enzyme molecule is directly connected to a positive and negative electrode to form the circuit. These circuits are capable of yielding highly informative signals of enzyme activity. These improved signals have greater signal-to-noise levels such that the signals are more distinguishable from noise, and these improved signals include features that carry detailed information about the engagement between enzyme and the target substrate.

In various embodiments, a molecular sensor comprises an enzyme-based molecular circuit (conductive pathway) such as described herein. Such a sensor having a polymerase enzyme is usable to sense sequence information from a DNA template processed by the polymerase.

In various embodiments of the present disclosure, a molecular circuit is disclosed. The circuit comprises: a positive electrode; a negative electrode spaced apart from the positive electrode; and an enzyme connected to both the positive and negative electrodes to form a conductive pathway between the positive and negative electrodes.

In various aspects, the enzyme of the circuit may comprise a first wiring point connected to the positive electrode and a second wiring point connected to the negative electrode.

In various aspects, the circuit may further comprise at least one arm molecule having first and second ends, the first end bonded to the enzyme and the second end bonded to at least one of the electrodes, wherein the at least one arm molecule acts as an electrical wire between the enzyme and at least one of the electrodes.

In various aspects, the at least one arm molecule may be selected from the group consisting of a double stranded oligonucleotide, a peptide nucleic acid duplex, a peptide nucleic acid-DNA hybrid duplex, a protein alpha-helix, a graphene-like nanoribbon, a natural polymer, a synthetic polymer, and an antibody Fab domain.

In various aspects, at least one of the electrodes is connected to an internal structural element of the enzyme.

In various aspects, the internal structural element may be selected from the group consisting of an alpha-helix, a beta-sheet, and a multiple of such elements in series.

In various aspects, at least one of the electrodes may be connected to the enzyme at a location of the enzyme capable of undergoing a conformational change.

In various aspects, at least one arm molecule may comprise a molecule having tension, twist or torsion dependent conductivity.

In various aspects, the enzyme may comprise a polymerase.

In various aspects, the polynerase comprises E. coli Pol I Klenow Fragment.

In various aspects, the polymerase comprises a reverse transcriptase.

In various aspects, the polymerase comprises a genetically modified reverse transcriptase.

In various aspects, a molecular sensor comprises a circuit further comprising a positive electrode; a negative electrode spaced apart from the positive electrode; and a polymerase enzyme comprising E. coli Pol I Klenow Fragment connected to both the positive and negative electrodes to form a conductive pathway between the positive and negative electrodes, wherein the positive electrode and the negative electrode each connect to the polymerase at connection points within the major alpha-helix of the polymerase extending between amino acids at position 514 and 547.

In various aspects, a molecular sensor comprises a circuit further comprising a positive electrode; a negative electrode spaced apart from the positive electrode; and a polymerase enzyme connected to both the positive and negative electrodes to form a conductive pathway between the positive and negative electrodes, wherein the sensor is usable to sense sequence information from a DNA template processed by the polymerase.

In various aspects, a molecular sensor comprises a circuit further comprising a positive electrode; a negative electrode spaced apart from the positive electrode; and a polymerase enzyme connected to both the positive and negative electrodes to form a conductive pathway between the positive and negative electrodes, wherein the positive electrode and the negative electrode each connect to the polymerase at connection points on the thumb and finger domains of the polymerase, and wherein such points undergo relative motion in excess of I nanometer as the polymerase processes a DNA template.

In various aspects, the polymerase in this sensor is engineered to have extended domains which produce a greater range of relative motion as the polymerase processes a DNA template.

In various aspects, the polymerase in this sensor is engineered to have additional charge groups that variably influence the internal conduction path as the enzyme processes a DNA template.

In various aspects, the polymerase in this circuit is a genetically modified form of E. coli. Pol I, Bst, Taq, Phi29, or T7 DNA polymerases, or a genetically modified reverse transcriptase.

In various aspects, a molecular circuit comprises: a positive electrode; a negative electrode spaced apart from the positive electrode; and an enzyme connected to both the positive and negative electrodes to form a conductive pathway between the positive and negative electrodes, wherein the positive electrode and the negative electrode each connect to the enzyme at connection points in the enzyme comprising at least one of a native cysteine, a genetically engineered cysteine, a genetically engineered amino acid with a conjugation residue, or a genetically engineered peptide domain comprising a peptide that has a conjugation partner.

In various aspects, this circuit further comprises a gate electrode.

In various embodiments, a method of sequencing a DNA molecule is disclosed. The method comprises: providing a circuit further comprising a positive electrode; a negative electrode spaced apart from the positive electrode; and a polymerase enzyme connected to both the positive and negative electrodes to form a conductive pathway between the positive and negative electrodes; initiating at least one of a voltage or a current through the circuit; exposing the circuit to a solution containing primed single stranded DNA and/or dNTPs; and measuring electrical signals through the circuit as the polymerase engages and extends a template, wherein the electrical signals are processed to identify features that provide information on the underlying sequence of the DNA molecule processed by the polymerase.

In various embodiments, a method of molecular detection is disclosed. The method comprises, providing a circuit further comprising: a positive electrode; a negative electrode spaced apart from the positive electrode; a polymerase enzyme connected to both the positive and negative electrodes to form a conductive pathway between the positive and negative electrodes and a gate electrode; initiating at least one of a voltage or a current through the circuit; exposing the circuit to at least one of: a buffer of reduced ionic strength, a buffer comprising modified dNTPs, a buffer comprising altered divalent cation concentrations, specific applied voltage on the primary electrodes, a gate electrode voltage, or voltage spectroscopy or sweeping applied to the primary electrodes or gate electrode; and measuring an electrical change in the circuit.

### BRIEF DESCRIPTION OF THE DRAWINGS

FIG. 1 provides an overview of immobilizing target proteins on sensor bridges to measure affinity reagent binding, or immobilizing specific affinity reagents (aptamers, nanobodies, antibodies) to capture target proteins (including biomarkers) meets the needs of both diagnostics and drug discovery. This Figure shows a DNA aptamer specific to COVID spike protein on bridge detecting recombinant S protein.

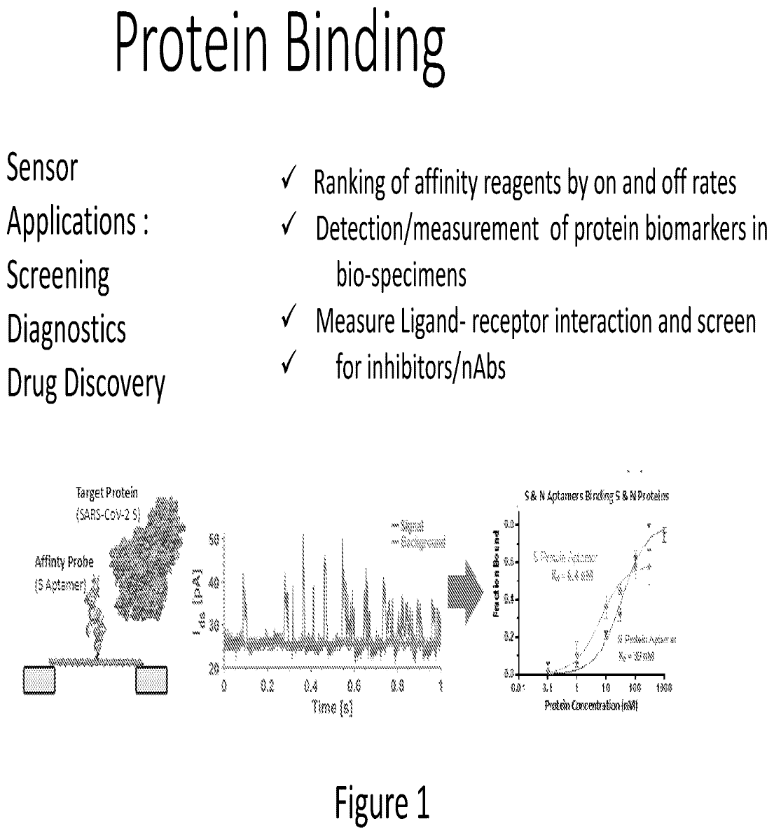

FIG. 2 illustrates that recombinant proteins are expressed with a terminal 6X Histidine tag that can be used for both purification and detection by anti-6X His antibodies. In this case a very high affinity anti-6XHis DNA aptamer was linked to the bridge as a general method to capture a broad range of recombinant protein targets. Once presented, the targets can subsequently capture specific affinity reagents including antibodies and nanobodies and the kinetics of their binding can be measured in real time on the chip.

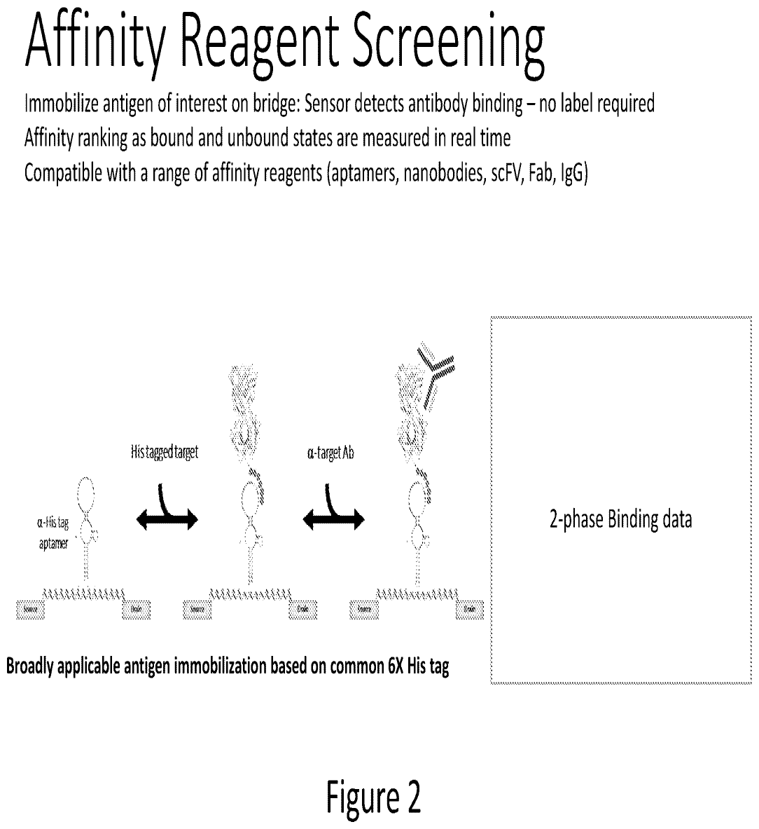

FIG. 3 illustrates the DNA aptamer is used to immobilize a recombinant COVID receptor binding domain (RBD) expressed with a 6X HIS tag and rmeasu ing the affinity of an anti-RBD antibody fragment (or nano body). This result is compatible with a range of affinity reagent including nanobody, full length ab or ab fragments,

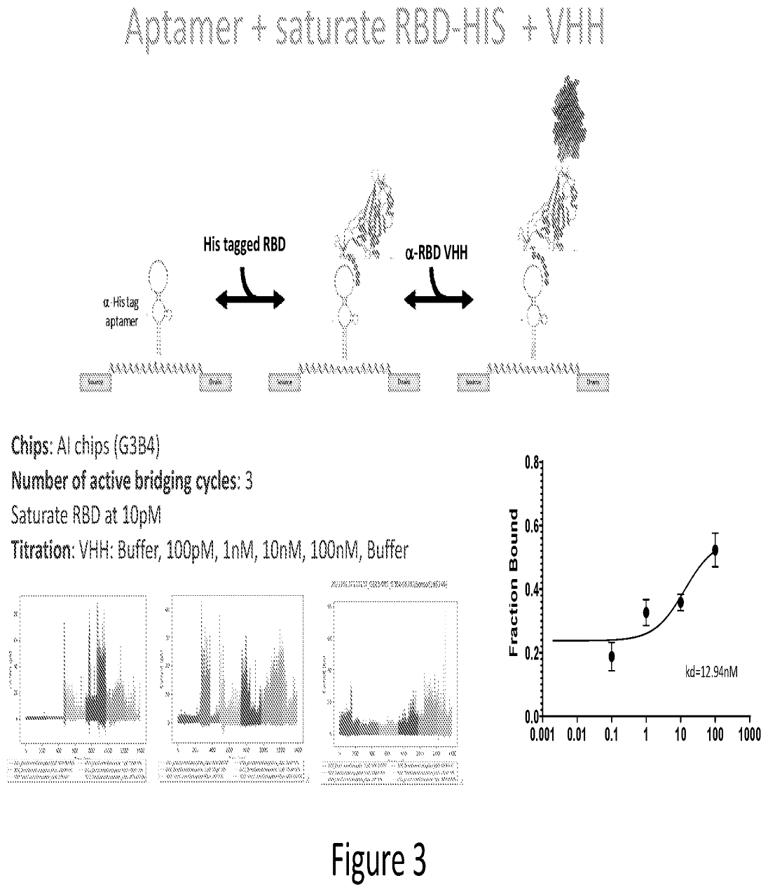

FIG. 4 illustrates the same anti 6x HIS aptamer from FIG. 3 used to immobilize a recombinant anti-RBD nanobody via 6X his tag. The results show binding affinity of aptarer is very high.

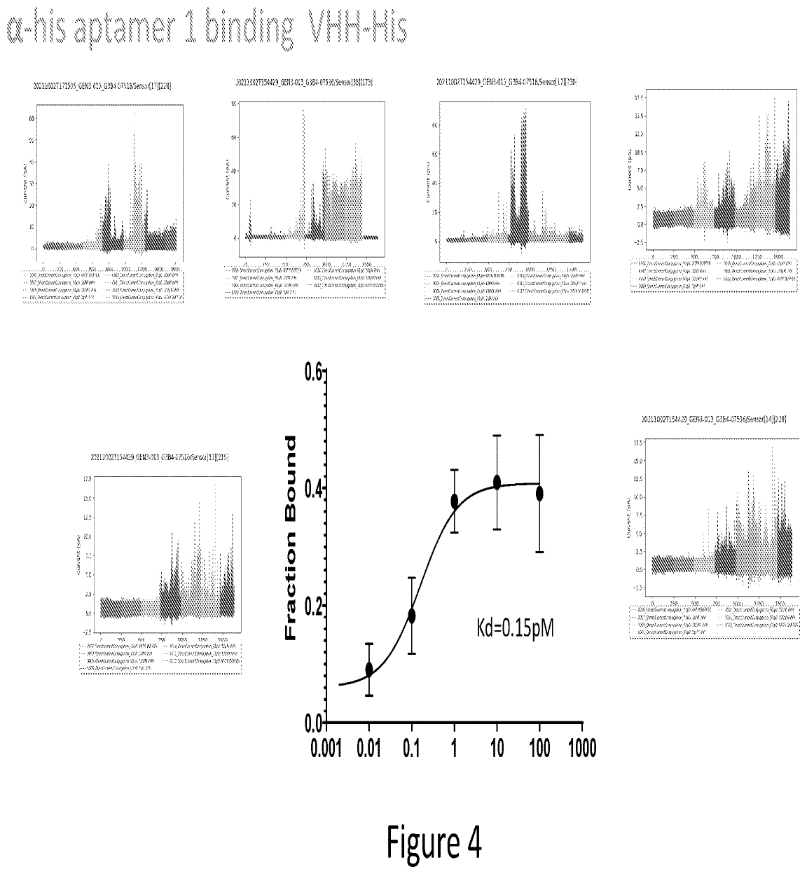

FIG. 5 shows the broad applicability of immobilization of antibodies using the 6X HIS tag. The measured affinity for RBD/VHH is very close regardless of which molecule is on the bridge.

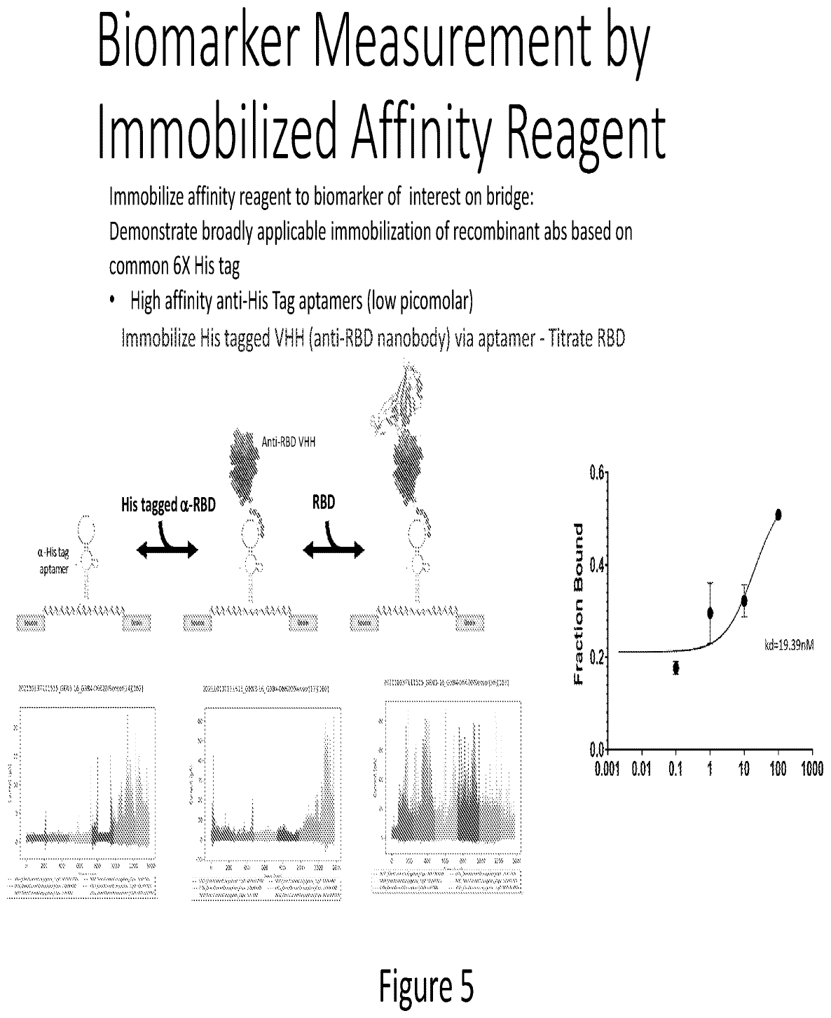

FIG. 6 shows that the aptarner is only one of several chemistries that can be employed to functionalize the bridges for protein binding and includes a lysine linkage, SpytagTxi/Spycatcher™, and detection can be performed by range of affinity reagents including nanobody or full-length IG.

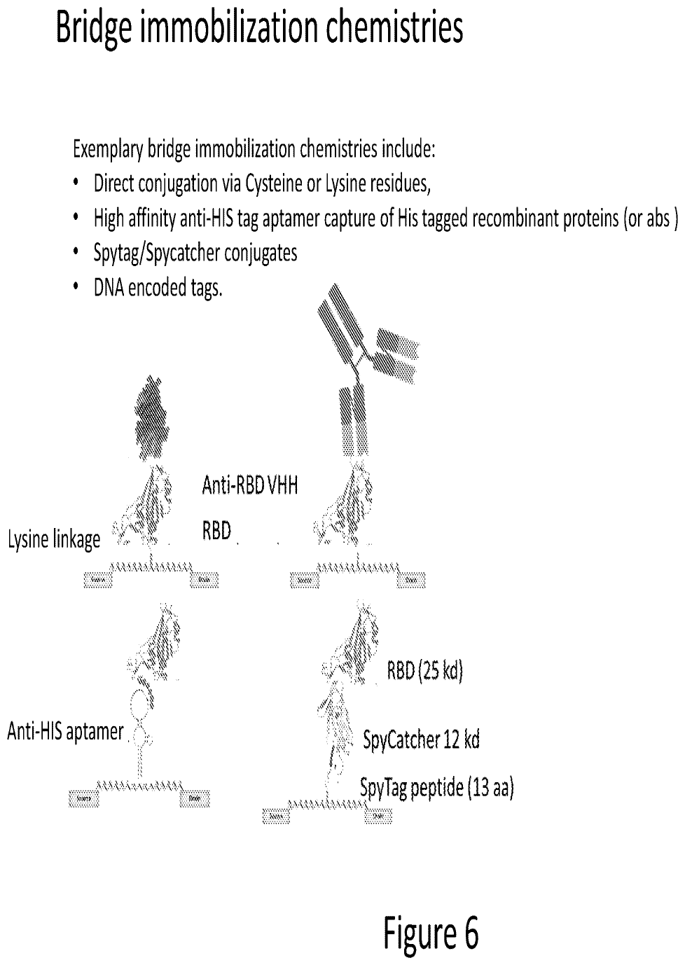

FIG. 7 illustrates an embodiment where the nanobody was immobilized via lysine chemistry and RBD binding was measured. The measured affinity remains in the same range as the other examples.

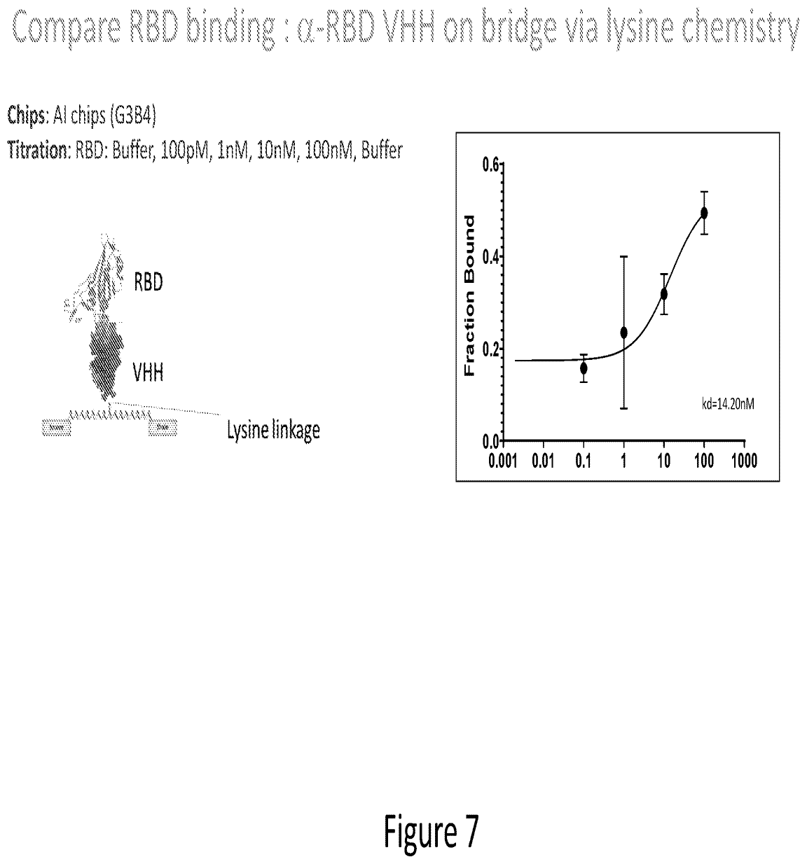

FIG. 8 illustrates the feasibility of drug screening through receptor binding and inhibition.

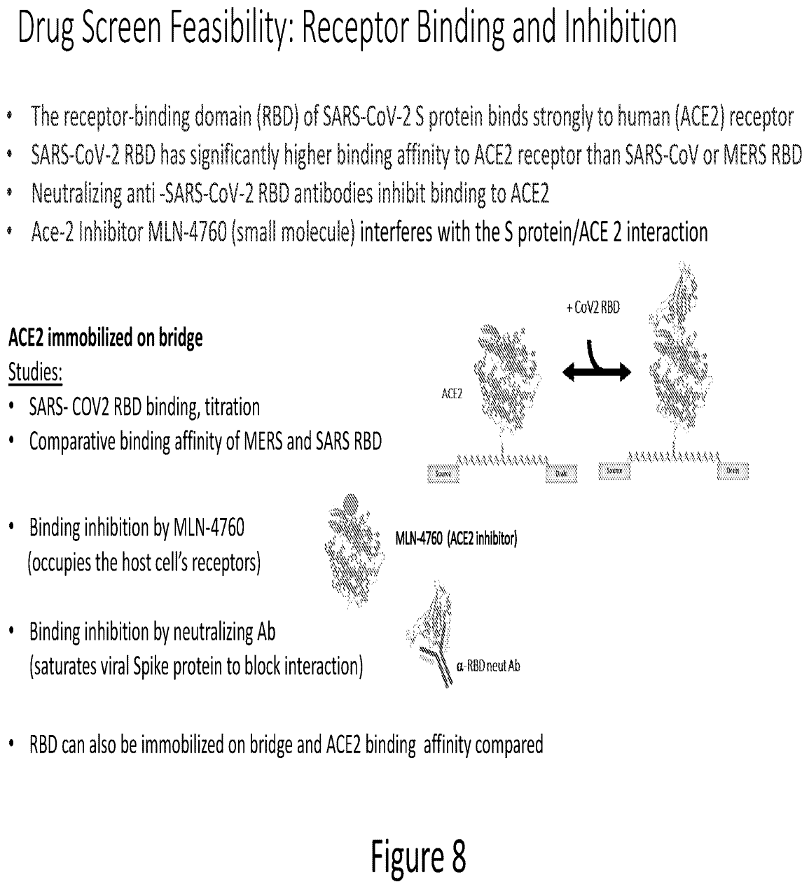

### DETAILED DESCRIPTION

Various aspects of the invention will now be described with reference to the following section which will be understood to be provided by way of illustration only and not to constitute a limitation on the scope of the invention.

### Definitions

As used herein, the term “enzyme” means a molecule that acts to transform another molecule, by engaging with a variety of substrate molecules. Such transformation could include chemical modification, or conformational modification. Common biological enzyme classes are polymerases, ligases, nucleases, kinases, transferases, as well as genetically modified forms of these molecules. Polymerases herein include reverse transcriptases and any genetically modified reverse transcriptase, capable of directly acting on an RNA template. Enzymes are most commonly proteins, but may be composed of multiple amino acid chains, and may also be complexed with other types of molecules, such as RNA in the case of the ribosome enzyme.

As used herein, the term “substrate” for an enzyme refers to any of the molecules that the enzyme specifically engages with in the course of performing a transformation. For example, in the specific case of a DNA polymerase, the substrate consists of both a template DNA and dNTPs. In addition to the substrates of the enzyme, the enzyme may also complex with various co-factors that moderate its function or kinetics. For example, in the case of DNA polymerase, divalent cations such as Mg++ are often essential cofactors, but not considered as substrates.

As used herein, the term “dNTP¹” or “dNTPs” refers to any of the deoxynucleotide triphosphates involved in polymerase-based DNA synthesis, or that can be engaged for such DNA synthesis, including both native and modified forms of such molecules.

As used herein, the term “buffer” for an enzyme refers to a solution in which the enzyme is viable and functional, and typically containing the substrates and co-factors needed for enzyme activity. Such an enzyme buffer may typically comprise salts, detergents, and surfactants, singly or in various combinations, as well as specific cofactors, such as magnesium or other divalent cations for a polymerase enzyme, along with the substrates, such as DNA and dNTPs for a polymerase enzyme, Such a buffer herein may have its composition modified from standard forms, such as to enhance signal properties in a sensor exposed to the buffer.

As used herein, the term “electrode” means any structure that can act as an efficient source or sink of charge carriers. Most commonly these would be metal or semiconductor structures, such as those used in electronic circuits. A pair of spaced apart electrodes herein may comprise a source and drain electrode pair. In various embodiments of the present disclosure, a binding probe-based molecular circuit may further comprise a gate electrode. When present, a gate electrode is used to apply a voltage rather than transfer charge carriers, Thus it supports accumulation of charge carriers to produce a local electric field, but is not intended to pass current, A gate electrode will be electrically isolated from the primary conduction paths of the circuit by some form of insulating layer or material.

As used herein, the term “conjugation” means any of the wide variety of means of physically attaching one molecule to another, or to a surface or particle. Such methods typically involve forming covalent or non-covalent chemical bonds, but may also rely on protein-protein interactions, protein-metal interactions, or chemical or physical adsorption via intermolecular (Vander Waals) forces. There is a large variety of such methods know to those skilled in the art of conjugation chemistry. Common conjugation methods relevant to preferred embodiments herein include thiol-metal bonds, maleimide-cysteine bonds, material binding peptides such as gold binding peptides, and click chemistries.

As used herein, the term “initiating,” in the context of an electrical parameter, is intended to be broader than the concept of “applying” an electrical value. For example, an electrical current may be initiated in a circuit, Such initiating of a current may be the result of applying a voltage to the circuit, but may be from other actions to the circuit besides applying a voltage. Further, a voltage may be initiated in a circuit. Such initiating of a voltage may be the result of applying a current to the circuit, but may be from other actions to the circuit besides applying an electrical current. In other examples, a voltage or a current may be initiated in one portion of a circuit as the result of applying a voltage or a current to the overall circuit. In a non-limiting example, a flow of electrons initiated from a negative to a positive electrode in a circuit of the present disclosure may be controlled by the voltage applied to the gate electrode of the circuit.

In various embodiments of the present disclosure, a molecular sensor comprises an enzyme connected to both a positive and a negative electrode to complete a circuit. Interactions of the enzyme with various substrates are detectable as changes in the current or other electrical parameter measured across the circuit. The present molecular differs from the general concept of a molecular electronic circuit in that the enzyme is directly “wired” to both the positive and negative electrodes rather than bonded to a molecular bridge molecule that spans the gap between the electrodes to complete a circuit.

In various aspects of the disclosure, at least one of a voltage or a current is initiated in an enzyme-based molecular circuit. When a target interacts with the enzyme, electrical changes in the circuit are sensed. These electrical changes, or informative electrical signals, may include current, voltage, impedance, conductivity, resistance, capacitance, or the like. In some examples, a voltage is initiated in the circuit and then changes in the current through the circuit are measured as substrates interact with the enzyme. In other examples, a current is initiated in the circuit, and changes to voltage in the circuit are measured as substrates interact with the enzyme. In other examples, impedance, conductivity, or resistance is measured. In examples wherein the circuit further comprises a gate electrode, such as positioned underneath the gap between the positive and negative electrodes, at least one of a voltage or current may be applied to the gate electrode, and voltage, current, impedance, conductivity, resistance, or other electrical change in the circuit may be measured as substrates interact with the enzyme.

TABLE 1
Summary of DATA from Figures

| | Immobilization | | | |
|---|---|---|---|---|
| Bridge | Chemistry | Target | Affinity | |
| Anti-his-aptamer-1 | Spytag/spycatcher | VHH -HIS | 0.1 | pM |
| Anti-his-aptamer-1 | Spytag/spycatcher | RBD-HIS | 0.4 | pM |
| VHH | lysine | RBD | 14.2 | nM |
| Anti-his-aptamer- | | Saturate VHH | 19.3 | nM |
| Spytag/spycatcher | | followed by RBD | | |
| 1 | | titration | | |
| Anti-his-aptamer-1 | Spytag/spycatcher | Saturate RBD | 12.9 | Nm |
| | | followed by VHH | | |
| | | titration | | |

Table 1 above shows anti his aptamer demoed similar affinity to 6xHis tag either on the VHH or the RBD. The measured affinity of the RBD/VHH interaction was quite close to the lit reported 20 nm and similar with different immobilization chemistry or which molecule was on bridge.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of, and” consisting of may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A sensor device comprising:
a first contact coupled to a first electrode;
a second contact coupled to a second electrode;
a sensor gap defined between one of the first contact and the first electrode and one of the second contact and the second electrode;
a bridge molecule comprising a first end and a second end; wherein the bridge molecule is coupled to the first contact at the first end and coupled to the second contact at the second end;
an aptamer that is coupled tot the bridge molecule; a binding partner that binds to the aptamer;
and wherein the binding interaction of the binding partner with the target ligand is detectable by the sensor.

2. The sensor device of claim 1, wherein the aptamer is specific for a tag on a binding partner.

3. The sensor device of claim 1, wherein the aptamer binds to a SARS COVID protein and the binding partner is SARS COVID protein.

4. The sensor device of claim 1, wherein the aptamer binds to the COVID-19 S protein and the binding partner is the COVID-19 S protein.

5. The sensor device of claim 1, wherein the aptamer comprises an a-His tag.

6. The sensor device of claim 1, wherein the binding partner selectively binds a target ligand.

7. The sensor device of claim 1, where the binding partner is an antibody or binding fragment thereof.

8. The sensor device of claim 1, wherein the aptamer comprises DNA.

9. The sensor device of claim 1, wherein the aptamer comprises a DNA that has affinity for the SARS CoV-2-S protein.

10. The sensor device of claim 1, wherein the aptamer comprises a DNA that has affinity for the 6Xhis tag on recombinant proteins including recombinant affinity reagents.

11. The sensor device of claim 1, wherein the aptamer comprises a peptide.

12. A method of identifying a binding partner, the method comprising:
i) selecting a sensor device;
ii) exposing the sensor to a solution comprising a target ligand of interest; and
iii) measuring the electrical signals to determine binding of the target to the binding partner.

13. The method of claim 12 used for drug screening.

14. The method of claim 12 used to detect or measure the binding of binding or an antibody or binding fragment thereof to a target ligand.
